(19) United States
(12) Patent Application Publication
(10) Pub. No.: US 2022/0194990 A1
(43) Pub. Date: Jun. 23, 2022

(54) VIRUS-LIKE PARTICLES AND METHODS OF USE THEREOF

(71) Applicant: Board of Regents of the University of Nebraska, Lincoln, NE (US)

(72) Inventors: Howard E. Gendelman, Omaha, NE (US); Jonathan Herskovitz, Omaha, NE (US); Mahmudul Hasan, Omaha, NE (US); Bhavesh Kevadiya, omaha, NE (US)

(21) Appl. No.: 17/310,245

(22) PCT Filed: Jan. 31, 2020
(86) PCT No.: PCT/US20/16126

§ 371 Date: Jul. 27, 2021

Related U.S. Application Data
(60) Provisional application No. 62/799,237, filed on Jan. 31, 2019.
(60) Provisional application No. 62/878,409, filed on Jul. 25, 2019.

(51) Int. Cl. C07K 14/005

(52) U.S. Cl. CPC C07K 14/005; C12N 2740/16042; C12N 2740/16023; C12N 2740/16022

(57) ABSTRACT

The present invention provides virus-like particles and methods of manufacture and use thereof. In accordance with the instant invention, virus-like particles (VLPs), particularly human immunodeficiency virus (HIV) VLPs, are provided. The HIV VLPs comprise at least one HIV structural protein and the HIV envelope protein, but lacks the HIV genome and lacks functional reverse transcriptase and integrase.

### CROSS-REFERENCE

This application is the U.S. National Phase of International Application No. PCT/US2020/016126, filed Jan. 31, 2020, which claims priority under 35 U.S.C. § 119(e) to U.S. Provisional Patent Application No. 62/799,237, filed Jan. 31, 2019 and U.S. Provisional Patent Application No. 62/878,409, filed Jul. 25, 2019. The foregoing applications are incorporated by reference herein in their entireties.

### STATEMENT AS TO FEDERALLY SPONSORED RESEARCH

This invention was made with government support under Grants Nos. P01 DA028555, R01 NS036126, P01 NS031492, R01 NS034239, P01 MH064570, P30 MH062261, P30 AI078498, R24 OD018546, R01 AG043540, and R01 AI145542 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jul. 27, 2021, is named EXV_011WOUS_SL.txt and is 3,959 bytes in size.

### FIELD OF THE INVENTION

The present invention relates generally to the delivery of therapeutics and/or imaging agents. More specifically, the present invention relates to compositions and methods for the delivery of therapeutic agents and/or imaging agents to a patient for the treatment or imaging of a viral infection.

### BACKGROUND OF THE INVENTION

According to UNAIDS, it is estimated that more than 36.7 million people worldwide are infected with the human immunodeficiency virus type one (HIV-1) and >5000 individuals worldwide are newly infected each day. In the clinic, antiretroviral therapy (ART) restricts viral infection by stalling various steps of the viral life cycle. However, ART fails to eliminate integrated copies of HIV-1 proviral DNA from the host genome (Chun, et al. (1997) Proc. Natl Acad. Sci., 94:13193-13197; Lorenzo-Redondo, et al. (2016) Nature 530:51-56). As such, virus persists in a latent state within infectious reservoirs; and ART cessation readily leads to viral reactivation and disease progression to acquired immunodeficiency syndrome (AIDS) (Deeks, et al. (2016) Nat. Med., 22:839-850). Thus, a major issue for any HIV-1 curative strategy is the means to eliminate either integrated proviral DNA or the cells that harbor virus without collateral cytotoxic reactions. However, elimination of HIV-1 infection in its infected human host is documented only in two individuals (Huffer, et al. (2009) New Engl. J. Med., 360:692-698; Gupta, et al. (2019) Nature 568:244-248). All single or combination therapeutic approaches preclude HIV-1 cure as viral rebound universally follows ART cessation (Li, et al. (2016) AIDS 30:343-353; Martin, et al. (2016) Annu. Rev. Med., 67:215-228; Saez-Cirion, et al. (2013) PLoS Pathog., 9:e1003211; Siliciano, et al. (2016) J. Clin. Investig., 126:409-414; Xu, et al. (2017) BioMed. Res. Int., 2017:6096134). There are several reasons why success has not yet been realized. This includes inadequate therapeutic access to viral reservoirs, rapid spread of infection by continuous sources of virus and susceptible cells and a failure to eliminate residual latent integrated proviral DNA. Therefore, improved methods of targeting HIV reservoirs are needed.

### SUMMARY OF THE INVENTION

In accordance with the instant invention, virus-like particles (VLPs), particularly human immunodeficiency virus (HIV) VLPs, are provided. The HIV VLPs comprise at least one HIV structural protein and the HIV envelope protein, but lacks the HIV genome and lacks functional reverse transcriptase and integrase. In certain embodiments, the VLP comprises HIV Gag, Pro, gp120, and gp41. The Gag in VLP may be cleaved at least into matrix and capsid. In certain embodiments, the HIV Env protein is dual-tropic for CXCR4 and CCR5, such as the Env from HIV-189.6. The VLP of the instant invention may comprise at least one therapeutic and/or at least one molecular imaging agent. In certain embodiments, the therapeutic and/or molecular imaging agent is biotinylated. In certain embodiments, the therapeutic and/or molecular imaging agent is conjugated to avidin, streptavidin, or analogue thereof such as monomeric streptavidin and its analogues. In certain embodiments, the therapeutic of the VLP is an anti-HIV agent. In certain embodiments, the therapeutic of the VLP is a CRISPR ribonucleoprotein, particularly wherein the guide RNA of the CRISPR ribonucleoprotein targets the HIV genome. In certain embodiments, the VLP comprises at least one anti-HIV agent and at least one CRISPR ribonucleoprotein. In certain embodiments, the VLP comprises a biotinylated HIV structural protein such as biotinylated matrix. In certain embodiments, the VLP comprises a HIV structural protein, such as capsid, conjugated to avidin, streptavidin, or an analogue thereof such as monomeric streptavidin or its analogues.

In accordance with another aspect of the instant invention, methods for synthesizing the VLP of the instant invention are provided.

In accordance with another aspect of the instant invention, methods of monitoring a viral infection are provided. In certain embodiments, the method comprises administering at least one VLP of the instant invention to a subject and detecting the presence and/or location of the molecular imaging agent of the VLP.

In accordance with another aspect of the instant invention, methods of treating, inhibiting, and/or preventing a viral infection are provided. In certain embodiments, the method comprises administering at least one VLP of the instant invention to a subject in need thereof. In certain embodiments, the viral infection is an HIV infection. The methods may further comprise administering at least one other anti-HIV agent to the subject.

### BRIEF DESCRIPTIONS OF THE DRAWING

FIG. 1 provides a schematic for the synthesis of virus-like particles (VLPs).

FIG. 2 provides a graph of p24 antigenicity as determined by enzyme-linked immunosorbent assay (ELISA) for different batches of VLPs.

FIG. 3A provides images of TZM-bl reporter cells stained with X-gal substrate which were uninfected (control) or infected with HIV-1 VLPs or two different quantities of HIV-1ADA. FIG. 3B provides a graph of the luminosity of TZM-bl reporter cells treated with D-luciferin which were uninfected (control) or infected with HIV-1 VLPs or two different quantities of HIV-1ADA.

FIGS. 4A-4D show that HIV-1 VLPs target HIV-1-infectable cells. Peripheral blood mononuclear cells (PBMCs) were cultured in the absence (untreated) or presence (treated) of IL2 and phytohemagglutinin (PHA) immune stimulant for 3 days and then treated with DiD fluorescently-labeled VLPs in biological triplicates for 24 hours. FIG. 4A provides a graph of the percent of gated populations positive for DiD fluorescent label (mean±SD). FIG. 4B provides a graph of the subpopulations normalized by relative abundance (mean±SD). Statistical analyses were performed using 2-way ANOVA in GraphPad Prism v7.0. *** p<0.001; ns: no statistical difference. Representative confocal microscopy images of unstimulated PBMCs treated with VLP-DiD followed by immunostaining anti-CD14-Alexa488 or anti-CD4-FITC antibodies for 30 minutes are provided in FIGS. 4C and 4D, respectively.

FIGS. 5A-5D show that HIV-1 VLPs target HIV-infectible cells in vivo. Human CD34+ hematopoietic stem-cell reconstituted NSG (humanized) mice were treated with VLP-DiD in triplicate. Blood (FIG. 5A) as well as single-cell suspensions from lymph nodes (FIG. 5B), liver (FIG. 5C), and spleen (FIG. 5D) were subjected to flow cytometry and the percent of gated populations (mean±SD) were graphed.

FIG. 6 provides images of real time biodistribution tests in humanized mice. Whole body single photon emission computed tomography computerized tomography (SPECT/CT) images were collected at 6, 24, 48 and 80 hours after intravenous injection of ¹⁷⁷Lu-CF-VLP and ¹⁷⁷Lu-CF NPs particles into a humanized mouse.

FIG. 7 provides an image of a PCR analysis of DNA extracted from CEM-SS T cells infected with HIV-1 and then treated with CRISPR-encoding plasmid (pCRISPR), VLPs (unloaded control), or CRISPR-delivering VLPs (VLP_{CRISPR}). Cells were also optionally treated with excess recombinant HIV-1 gp120. Uninfected and untreated controls are also provided. 1: unexcised proviral HIV-1 DNA; 2: positive control for size of PCR product when proviral HIV-1 excised; 3: PCR band after HIV-1 proviral excision.

### DETAILED DESCRIPTION OF THE INVENTION

In accordance with the instant invention, virus-like particles (VLPs), particularly human immunodeficiency virus (HIV) VLPs (e.g., HIV-1 VLPs), are provided. VLPs of the instant invention are biomimetics of a virus and have a structure resembling a virus particle, particularly HIV, but which are not pathogenic and are not replication competent. The VLPs of the instant invention lack the viral genome. Generally, the VLPs lack any genetic information encoding for the proteins of the VLP, but may contain nucleic acid molecules distinct from the viral genome (e.g., therapeutic nucleic acid molecules). The VLPs of the instant invention will generally be the same size as a virion particle, e.g., an HIV particle. For example, the diameter or longest dimension of the VLP may be about 10 to about 500 nm, about 50 nm to about 400 nm, about 50 nm to about 300 nm, about 50 nm to about 200 nm, about 75 nm to about 200 nm, about 75 nm to about 150 nm, or about 90 nm to about 150 nm. The VLPs are typically round or spherical shaped. The VLPs of the instant invention may be used as targeting vector for the delivery of any compound, particularly one or more therapeutics (e.g., antiviral or antiretroviral therapeutics, gene editing tools, and the like) and/or one or more molecular imaging agents.

The VLP of the instant invention demonstrate improved targeting and delivery of therapeutics to monocyte/macrophage and CD4+ T cell populations. Improved targeting resulted in enhanced uptake and improved antiviral responses in CD4+ T cells and monocyte/macrophage cells, which are the principal targets of HIV (e.g., HIV-1) infection. Furthermore, in vitro treatment of elutriated human peripheral blood mononuclear cell (PBMC) with VLPs of the instant invention (e.g., labeled with fluorescent DiD) resulted in high levels of CD4+ T cell localization, which was significantly boosted upon IL-2 and PHA stimulation. This trend was absent in monocytes/macrophages and CD19+ B cells. Together, the data presented herein demonstrate that VLP target the same populations of cells as replication competent HIV-1 in scenarios of immune-activation. As seen in the Example, VLP-targeting was confirmed in humanized mice, with 84.2% of CD14+ monocytes/macrophages and 36.6% of CD4+ T cells positive for fluorescent DiD signal 24-hours post-treatment of VLPs, with limited off-target signal emanating from CD8+ T cells and murine huCD45− cells.

In order to increase loading capacity of VLPs, the VLPs of the instant invention may also comprise avidin/streptavidin or analogues thereof loading sites or biotin loading sites, such as below HIV envelope or the lipid membrane, to retain the capacity for intrinsic loading of any biotinylated payload or avidin/streptavidin or analogue thereof conjugated payload. This arrangement allows for the VLPs to carry sufficient amounts of the antiviral payloads while being able to release the payloads upon reaching its destination in virus containing tissues and cells.

The VLPs of the instant have multiple advantages to other delivery systems. The VLPs of the instant invention can comprise a viral envelope capable of targeting two cellular subtypes (CD4+ T cells and monocytes/macrophages) with exceptionally high targeting capacity, which confers maximum range of on-target therapeutic and/or diagnostic delivery to HIV-1 infectible cells and virus-infected tissue compartments and/or reservoirs of infection. Moreover, the VLPs of the instant invention allow for targeting of any compound to the desired cells such as therapeutic agents (e.g., antiretroviral therapeutics drugs and/or gene editing tools (e.g., gene therapy)) and/or molecular imaging agents (e.g., SPECT/CT/PET imaging agents). In certain embodiments, the VLPs comprise one or more antiretroviral therapeutics drug, one or more gene editing tool (e.g., CRISPR), and one or more molecular imaging agents. Additionally, the VLPs of the instant invention can be generated by plasmid co-transfection (optionally stably) into highly expressive cell systems, thereby enabling the scalable manufacturing of the VLPs. The simple loading scheme is also superior compared to other lentiviral vectors that require individual fusion protein constructs for protein loading. Moreover, as explained above, the VLPs possess broader payload material carrying capacity that can include small molecules, proteins, and nucleotide-protein complexes. Lastly, the VLPs of the instant invention possess improved safety profiles compared to other viral vectors due to replication incompetence and absence of key viral proteins and enzymes. The VLPs, through its theranostic capabilities, may also be utilized to monitor, prophylactically protect, treat, and cure organisms from potential future exposure to or actively ongoing HIV-1 infection.

In certain embodiment, the VLPs of the instant invention comprise the structural proteins of HIV. In certain embodiments, the VLPs may comprise Gag (p55; e.g., PubMed Gene ID: 155030), Pol, and Pro (protease). Gag is a precursor protein which associates with the cytoplasmic side of cell membranes and triggers the budding of the viral particle from the surface of an infected cell. Gag is cleaved by Pro during viral maturation into four smaller proteins: matrix (MA; p17), capsid (CA; p24), nucleocapsid (NC; p7), and p6. MA largely remains attached to the inner surface of the virion lipid bilayer, thereby stabilizing the particle. CA forms the conical core of viral particles. NC recognizes the packaging signal of HIV RNA and p6 interacts with accessory protein Vpr, leading to its incorporation of into virions. Pol, which is cleaved from a Gag-Pol precursor (e.g., PubMed Gene ID: 155348), is also cleaved by Pro to protease (p10), reverse transcriptase (RT; p50), RNase H (p15), and integrase (p31). However, the VLPs of the instant invention lack reverse transcriptase and integrase or functional versions of both. The lack of functional reverse transcriptase and integrase prevents transgene insertion in host cell nuclei. Thus, the VLP-encoding constructs contain full-length Gag and a modified Pol containing protease, but lacking RT, RNase H, and integrase.

In certain embodiment, the VLPs of the instant invention also comprise HIV envelope protein (Env; e.g., PubMed Gene ID: 155971). Env is expressed as precursor in cells which is cleaved into a transmembrane domain (gp41) and an extracellular domain (gp120). Env mediates entry of virions into cells through interaction with its receptor CD4 and various co-receptors. In a particular embodiment, the Env is dual-tropic for CCR5 and CXCR4. In a particular embodiment, the Env is from HIV-1. In a particular embodiment, the Env is from a dual macrophage and lymphocyte-tropic strain of HIV-1. In a particular embodiment, the Env is from HIV-189.6 (e.g., GenBank Accession No. AAA81043.2). Being dual-tropic enables VLPs to target all major HIV-1 cellular targets/reservoirs which include both CD4+ effector memory and regulatory T cells as well as mononuclear phagocytes (MP; monocytes, macrophages and dendritic cells) using the CCR5 co-receptor and broad populations of CD4+ T cells (using the CXCR4 co-receptor). In certain embodiments, the VLP comprises gp41 and gp120. In certain embodiments, the VLP comprises gp160, gp41, and gp120.

The VLPs may comprise one or both of HIV regulatory proteins: Tat and Rev. Generally, the VLPs lack any HIV regulatory proteins. Similarly, the VLPs may comprise, 1, 2, 3, or 4 HIV accessory proteins: Vpu, Vpr, Vif, and Nef. Generally, the VLPs lack any HIV accessory proteins.

In certain embodiments, the VLP comprises the HIV structural proteins and Env. In certain embodiment, the VLP comprises Gag, Pro, Pol, gp120, gp41, and gp160. In certain embodiment, the VLP comprises Gag, Pro, Pol, gp120, and gp41. In certain embodiments, the Pol is a modified comprising protease, but lacking RT, RNase H, and integrase. In certain embodiment, the VLP comprises Gag, Pro, gp120, gp41, and gp160. In certain embodiment, the VLP comprises Gag, Pro, gp120, and gp41. In certain embodiments, the Gag, Pro, and Pol are those encoded by psPAX2. The VLPs may comprise Gag or, if cleaved by Pro during viral maturation, the VLPs may comprise matrix (MA; p17), capsid (CA; p24), nucleocapsid (NC; p7), and p6.

In certain embodiments, at least one protein, particularly at least one structural protein, of the VLP comprises biotin. In certain embodiments, the biotinylated protein is matrix (p17). In a particular embodiment, the matrix protein of the VLP is a fusion of the HIV-1 matrix (p17) and an amino acid sequence biotinylated by the E. coli biotin ligase, BirA. In a particular embodiment, the matrix protein of the VLP is a fusion of the HIV-1 matrix (p17) and the AviTag™ sequence (e.g., at the N-terminus or C-terminus of the matrix protein, particularly the C-terminus). In a particular embodiment, the matrix protein of the VLP is a fusion of the HIV-1 matrix (p17) and an amino acid sequence comprising GLNDIFEAQKIEWHE (SEQ ID NO: 1) (e.g., at the N-terminus or C-terminus of the matrix protein, particularly the C-terminus). The p17::Avi fusion is readily biotinylated in the presence of BirA biotin ligase (e.g., BirA biotin ligase may be expressed during VLP synthesis), enabling conjugation to avidin/streptavidin containing therapeutics or molecular imaging agents (e.g., streptavidin quantum dot fluorescent probes). Notably, for synthesis of the fusion protein, restriction enzyme sites may be inserted into the nucleotide sequence encoding AviTag™ (e.g., a silent T15A substitution to clone in an EcoRV digestion site), to facilitate cloning into the fusion protein. The exposed biotin of the biotinylated matrix serves as a ligand for avidin/streptavidin binding, thereby attaching a therapeutic agent or molecular imaging agent comprising avidin, streptavidin, or an analogue thereof to the spherical matrix underlying the envelope of the VLP.

In certain embodiments, at least one protein, particularly at least one structural protein, of the VLP comprises avidin, streptavidin, or an analogue thereof. In a particular embodiment, the VLP protein comprises monomeric streptavidin or an analogue thereof. Examples of amino acid sequences for monomeric streptavidin include:

(SEQ ID NO: 2)
EFASAEAGITGTWYNQHGSTFTVTAGADGNLTGQYENRAQGTGCQNSPYT
LTGRYNGTKLEWRVEWNNSTENCHSRTEWRGQYQGGAEARINTQWNLTY
EGGSGPATEQGQDTFTKVKPSAASGS
and
(SEQ ID NO: 3; Lim et al. (2013) Biotechnol.
Bioeng., 110: 57-67)
AEAGITGTWYNQSGSTFTVTAGADGNLTGQYENRAQGTGCQNSPYTLTGR
YNGTKLEWRVEWNNSTENCHSRTEWRGQYQGGAEARINTQWNLTYEGGS
GPATEQGQDTFTKVK.

In a particular embodiment, the VLP protein comprises maxavidin, which comprises:

(SEQ ID NO: 4)
EFASAEAGITGTWYNQSGSTFTVTAGADGNLTGQYENRAQGTGCQNSPYT
LTGRYNGTKLEWRVEWNNSTENCHSRTEWRGQYQGGAEARINTQWNLTYE
GGSGPATEQGQDTFTKVKPSAASGS.

Maxavidin is a monomeric streptavidin analogue constructed with very high affinity for biotin or biotin-conjugates, high stability, and low interference with the three-dimensional structure of the fused protein. In certain embodiments, the VLP protein comprising avidin, streptavidin, or an analogue thereof (monomeric streptavidin or an analogue thereof) is capsid (p24). In a particular embodiment, the capsid protein of the VLP is a fusion of the HIV-1 capsid (p24) and the amino acid sequence of a monomeric streptavidin or an analogue thereof (e.g., at the N-terminus or C-terminus of the capsid protein, particularly the C-terminus). p24::maxavidin fusion proteins comprising the VLP capsid offer many binding sites for the capture of biotinylated payloads (e.g., therapeutics (e.g., antiretroviral drugs and/or HIV-1 inactivating endonuclease ribonucleoproteins) and or molecular imaging agents (e.g., tracking probes). Biotinylated payloads (e.g., therapeutics and/or molecular imaging agents) may be pre-loaded into VLP producer cells. Upon VLP synthesis, biotinylated payloads bind p24::maxavidin and become incorporated to the conical capsid core.

Biotin may be attached to the therapeutics and/or molecular imaging agents either directly or via a linker or chemical spacer. Generally, the linker is a chemical moiety comprising a covalent bond or a chain of atoms that covalently attaches the biotin to the therapeutic and/or molecular imaging agent. The linker can be linked to any synthetically feasible position. Exemplary linkers may comprise at least one optionally substituted; saturated or unsaturated; linear, branched or cyclic aliphatic group, an alkyl group, or an optionally substituted aryl group. The linker may be a lower alkyl or aliphatic. The linker may also be a polypeptide (e.g., from about 1 to about 10 amino acids, particularly about 1 to about 5). The linker may be degradable or hydrolysable in a cell such that it is substantially cleaved or completely cleaved. In a particular embodiment, the linker or chemical spacer is cleavable by cellular enzymes (e.g., esterases, thioreductases, amidases, cathepsins (e.g., cathepsin K), MMPs, and the like) or is pH sensitive, particularly wherein the linker or chemical spacer is cleaved under acidic conditions (e.g., pH<6, particularly <5.5). In a particular embodiment, the linker comprises at least one hydrazone bond, acetal bond, cis-aconityl spacer, phosphamide bond, and/or silyl ether bond.

Notably, both matrix (p17) and capsid (p24) are cleaved from a polyprotein (Gag) during VLP maturation (i.e., assembly and budding from producer cells). Thus, material payloads are retained intrinsically while preserving the natural targeting of HIV envelope.

As stated hereinabove, the VLPs of the instant invention may further comprise one or more therapeutics (e.g., antiviral or antiretroviral therapeutics, gene therapy or gene editing tools, etc.) and/or molecular imaging agents. In certain embodiments, the therapeutics and/or molecular imaging agents are biotinylated or conjugated/fused to avidin/streptavidin or analogues thereof. For example, the VLP may comprise biotinylated fluorophores, biotinylated antiretroviral drugs or prodrugs, and/or biotinylated CRISPR/Cas9 ribonucleoproteins.

Therapeutics of the instant invention include, but are not limited to: small molecules, peptides, proteins, nucleoside and nucleotide analogs, prodrugs, nanoformulated drugs (such as nanoformulated antiretroviral compounds), and DNA and/or RNA based molecules such as siRNAs, miRNAs, antisense, and CRISPR/Cas9 constructs (e.g., for gene therapy). In certain embodiments, the therapeutic compound is an antiviral or an antiretroviral. In a particular embodiment, the therapeutic compound is rilpivirine or biotinylated rilpivirine. In a particular embodiment, the therapeutic compound is cabotegravir or biotinylated cabotegravir. The antiretroviral may be effective against or specific to lentiviruses. Lentiviruses include, without limitation, human immunodeficiency virus (HIV) (e.g., HIV-1, HIV-2), bovine immunodeficiency virus (BIV), feline immunodeficiency virus (FIV), simian immunodeficiency virus (SIV), and equine infectious anemia virus (EIA). In a particular embodiment, the therapeutic agent is an anti-HIV agent. An anti-HIV compound or an anti-HIV agent is a compound which inhibits HIV (e.g., inhibits HIV replication and/or infection). Examples of anti-HIV agents include, without limitation:

(I) Nucleoside-analog reverse transcriptase inhibitors (NRTIs). NRTIs refer to nucleosides and nucleotides and analogues thereof that inhibit the activity of reverse transcriptase, particularly HIV-1 reverse transcriptase. NRTIs comprise a sugar and base. Examples of nucleoside-analog reverse transcriptase inhibitors include, without limitation, adefovir dipivoxil, adefovir, lamivudine, telbivudine, entecavir, tenofovir, stavudine, abacavir, didanosine, emtricitabine, zalcitabine, and zidovudine.

(II) Non-nucleoside reverse transcriptase inhibitors (NNRTIs). NNRTIs are allosteric inhibitors which bind reversibly at a nonsubstrate-binding site on reverse transcriptase, particularly the HIV reverse transcriptase, thereby altering the shape of the active site or blocking polymerase activity. Examples of NNRTIs include, without limitation, delavirdine (DLV, BHAP, U-90152; Rescriptor®), efavirenz (EFV, DMP-266, SUSTIVA®), nevirapine (NVP, Viramune®), PNU-142721, capravirine (S-1153, AG-1549), emivirine (+)-calanolide A (NSC-675451) and B, etravirine (ETR, TMC-125, Intelence®), rilpivirne (RPV, TMC278, Edurant™) DAPY (TMC120), doravirine (Pifeltro™), BILR-355 BS, PHI-236, and PHI-443 (TMC-278).

(III) Protease inhibitors (PI). Protease inhibitors are inhibitors of a viral protease, particularly the HIV-1 protease. Examples of protease inhibitors include, without limitation, darunavir, amprenavir (141W94, AGENERASE®), tipranivir (PNU-140690, APTIVUS®), indinavir (MK-639; CRIXIVAN®), saquinavir (INVIRASE®, FORTOVASE®), fosamprenavir (LEXIVA®), lopinavir (ABT-378), ritonavir (ABT-538, NORVIR®), atazanavir (REYATAZ®), nelfinavir (AG-1343, VIRACEPT®), lasinavir (BMS-234475/CGP-61755), BMS-2322623, GW-640385X (VX-385), AG-001859, and SM-309515.

(IV) Fusion or entry inhibitors. Fusion or entry inhibitors are compounds, such as peptides, which block HIV entry into a cell (e.g., by binding to HIV envelope protein and blocking the structural changes necessary for the virus to fuse with the host cell). Examples of fusion inhibitors include, without limitation, CCR5 receptor antagonists (e.g., maraviroc (Selzentry®, Celsentri)), enfuvirtide (INN, FUZEON®), T-20 (DP-178, FUZEON®) and T-1249.

(V) Integrase inhibitors (integrase-strand transfer inhibitors (INSTIs)). Integrase inhibitors are a class of antiretroviral drug designed to block the action of integrase (e.g., HIV integrase), a viral enzyme that inserts the viral genome into the DNA of the host cell. Examples of integrase inhibitors include, without limitation, cabotegravir (CAB), raltegravir (RAL), elvitegravir (EVG), dolutegravir (DTG), bictegravir (BIC), BI 224436, and MK-2048.

Anti-HIV compounds also include maturation inhibitors (e.g., bevirimat). Maturation inhibitors are typically compounds which bind HIV Gag and disrupt its processing during the maturation of the virus. Anti-HIV compounds also include HIV vaccines such as, without limitation, ALVAC® HIV (vCP1521), AIDSVAX®B/E (gp120), and combinations thereof. Anti-HIV compounds also include HIV antibodies (e.g., antibodies against gp120 or gp41), particularly broadly neutralizing antibodies.

More than one anti-HIV agent may be used, particularly where the agents have different mechanisms of action (as outlined above). For example, anti-HIV agents which are not NNRTIs may be combined with NNRTI drugs. In a particular embodiment, the anti-HIV agents include agents used in highly active antiretroviral therapy (HAART).

The therapeutic can be a prodrug or nanoformulated drug. Examples of prodrugs and nanoformulated drugs include long acting formulations of anti-retrovirals and include those described in PCT/US2019/063498, PCT/US2019/057406, WO 2019/199756, WO 2019/140365, U.S. patent application Ser. No. 16/304,759, each of the foregoing incorporated by reference herein.

In certain embodiments, the therapeutic is a gene editing tool. In certain embodiments, the VLPs of the instant invention comprise at least one gene editing tool. The therapeutic may be a gene editing tool to excise or delete all or part of the viral genome within a cell, particularly the HIV-1 genome, particularly the integrated HIV-1 genome. The viral genome can be edited, excised, or deleted using any method known in the art such as, without limitation: zinc finger nucleases (ZFNs), transcription activator like effector nucleases (TALENs), clustered regularly interspaced short palindromic repeats (CRISPRs), and meganucleases. In certain embodiments, CRISPR is utilized. Clustered, regularly interspaced, short palindromic repeat (CRISPR)/Cas9 (e.g., from Streptococcus pyogenes) technology and gene editing are well known in the art (see, e.g., Shi et al. (2015) Nat. Biotechnol., 33(6):661-7; Sander et al. (2014) Nature Biotech., 32:347-355; Jinek et al. (2012) Science, 337:816-821; Cong et al. (2013) Science 339:819-823; Ran et al. (2013) Nature Protocols 8:2281-2308; Mali et al. (2013) Science 339:823-826; Sapranauskas et al. (2011) Nucleic Acids Res. 39:9275-9282; Nishimasu et al. (2014) Cell 156(5):935-49; Swarts et al. (2012) PLoS One, 7:e35888; Sternberg et al. (2014) Nature 507(7490):62-7; addgene.org/crispr/guide). The RNA-guided CRISPR/Cas9 system involves using Cas9 along with a guide RNA molecule (gRNA). Guidelines and computer-assisted methods for generating gRNAs are available and well known in the art (see, e.g, CRISPR Design Tool (crispr.mit.edu); Hsu et al. (2013) Nat. Biotechnol. 31:827-832; addgene.org/CRISPR; and CRISPR gRNA Design tool—DNA2.0 (dna20.com/eCommerce/startCas9)). gRNAs bind and recruit Cas9 to a specific target sequence (e.g., viral genome) where it mediates a double strand DNA (dsDNA) break. More than one gRNA (e.g., two) may be administered to make multiple breaks within the target nucleic acid. The double strand break can be repaired by non-homologous end joining (NHEJ) pathway yielding a deletion of the target nucleic acid. While CRISPR is described herein as utilizing Cas9, other nucleases such as Cas9 variants and homologs can be used. Other examples include, without limitation, Streptococcus pyogenes Cas9, Cas9 D10A, high fidelity Cas9 (Kleinstiver et al. (2016) Nature, 529:490-495; Slaymaker et al. (2016) Science, 351:84-88), Cas9 nickase (Ran et al. (2013) Cell, 154:1380-1389), Streptococcus pyogenes Cas9 with altered PAM specificities (e.g., SpCas9_VQR, SpCas9 EQR, and SpCas9_VRER; Kleinstiver et al. (2015) Nature, 523:481-485), Staphylococcus aureus Cas9, cas12a (Cpf1) (Rusk, N., Nat. Methods (2019) 16(3):215), the CRISPR/Cpf1 system of Acidaminococcus, and the CRISPR/Cpf1 system of Lachnospiraceae.

The binding specificity of the CRISPR/Cas9 complex depends on two different elements. First, the binding complementarity between the targeted sequence (e.g., viral genome) and the complementary recognition sequence of the gRNA (e.g., ˜18-22 nucleotides, particularly about 20 nucleotides). Second, the presence of a protospacer-adjacent motif (PAM) juxtaposed to the target DNA/gRNA complementary region (Jinek et al. (2012) Science 337:816-821; Hsu et al. (2013) Nat. Biotech., 31:827-832; Sternberg et al. (2014) Nature 507:62-67). The PAM motif for S. pyogenes Cas9 has been fully characterized, and is NGG or NAG (Jinek et al. (2012) Science 337:816-821; Hsu et al. (2013) Nat. Biotech., 31:827-832). Other PAMs of other Cas9 proteins are also known (see, e.g., addgene.org/crispr/guide/#pam-table). Typically, the PAM sequence is 3′ of the target sequence in the genomic sequence.

The guide RNA may comprise separate nucleic acid molecules wherein one RNA may specifically hybridize to a target sequence (crRNA) and another RNA (trans-activating crRNA (tracrRNA)) specifically hybridizes with the crRNA. Preferably, the guide RNA is a single molecule (sgRNA) which comprises a sequence which specifically hybridizes (e.g., complete complementary) with a target sequence (crRNA; complementary sequence) and a sequence recognized by Cas9 (e.g., a tracrRNA sequence; scaffold sequence), which are well known in the art. The greater the complementarity reduces the likelihood of undesired cleavage events at other sites of the genome. In a particular embodiment, the region of complementarity (e.g., between a guide RNA and a target sequence) is at least about 10, at least about 12, at least about 15, at least about 17, at least about 20, at least about 25, at least about 30, at least about 35, or more nucleotides. In a particular embodiment, the region of complementarity (e.g., between a guide RNA and a target sequence) is about 15 to about 25 nucleotides, about 15 to about 23 nucleotides, about 16 to about 23 nucleotides, about 17 to about 21 nucleotides, about 18 to about 22 nucleotides, or about 20 nucleotides. In a particular embodiment, the guide RNA targets a sequence or comprises a sequence (e.g., RNA version) which has at least 80%, 85%, 90%, 95%, 97%, 99%, or 100% homology or identity to the target sequence. In certain embodiments, the gRNA targets (and inactivates or deletes) all or part of integrated HIV-1 DNA. In certain embodiments, the gRNA targets (and inactivates or deletes) all or part of the transactivator of transcription (tat) gene. In certain embodiments, at least two different gRNA are used. For example, one gRNA may target the transactivator of transcription (tat) gene and the other gRNA may target another region of the integrated HIV-1 genome (e.g., a region other than LTR). In a particular embodiment, at least one of the CRISPR and gRNA are selected from those described in Dash et al. (Nat. Comm. (2019) 10(1):2753), incorporated by reference herein.

CRISPR can be incorporated into VLPs in various ways. In certain embodiments, at least one Cas9 (e.g., the protein and/or a nucleic acid molecule encoding Cas9) and at least one gRNA or a nucleic acid molecule encoding the gRNA can be delivered to the VLP producing cell. In a particular embodiment, the Cas9 is S. pyogenes Cas9. In certain embodiments, one or more (e.g., two) ribonucleoprotein comprising Cas9 and a gRNA is delivered to the VLP producing cell, optionally wherein the ribonucleoprotein(s) is biotinylated or conjugated/fused to avidin, streptavidin, or analogue thereof.

Molecular imaging agents (e.g., diagnostic agents) may also be contained with the VLPs, optionally with a therapeutic agent. In certain embodiments, the molecular imaging agent is detectable by flow cytometry, single-photon emission computed tomography/computed tomography (SPECT/CT) radiography, positron emission tomography (PET), in vivo imaging system (IVIS), confocal microscopy imaging, or magnetic resonance imaging (MRI). Examples of molecular imaging agents include, without limitation: optical imaging agents (e.g., near IR dyes (e.g., IRDye 800CW), phorphyrins, anthraquinones, anthrapyrazoles, perylenequinones, xanthenes, cyanines, acridines, phenoxazines, phenothiazines and derivatives thereof), fluorescent compounds (e.g., Alexa Fluor® dyes (e.g., Alexa Fluor® 488), fluorescein, rhodamine, Cy3, Cy5, DiI, DiO, DID and derivatives thereof), chromophores, paramagnetic or superparamagnetic ions (e.g., Gd(III), Eu(III), Dy(III), Pr(III), Pa(IV), Mn(II), Cr(III), Co(III), Fe(III), Cu(II), Ni(II), Ti(III), and V(IV)), magnetic resonance imaging (MRI) contrast agents (e.g., heavy metals, DOTA-Gd3+, DTPA-Gd3+ (gadolinium complex with diethylenetriamine pentaacetic acid)), positron emission tomography (PET) agents (labeled or complexed with ¹¹C, ¹³N, ¹⁵O, ¹⁸F, ⁶⁴Cu, ⁶⁸Ga, or ⁸²Rb (e.g., (fluorodeoxyglucose))), computerized tomography (CT) agents (e.g., iodine or barium containing compounds, e.g., 2,3,5-triiodobenzoic acid), gamma or positron emitters (e.g., ^{99m}Tc, ¹¹¹In, ¹¹³In, ¹⁵³Sm, ¹²³I, ¹³¹I, ¹⁸F, ⁶⁴Cu, ¹⁷⁷Lu ²⁰¹Tl, etc., optionally complexed to other compounds (e.g., metal particles), radioisotopes, isotopes, biotin, gold (e.g., nanoparticles), radiolabeled compounds (e.g., radiolabeled nanoparticles), metal particles or nanoparticles (e.g., iron oxide, cobalt ferrite, CuS, quantum dots (QDs), Bismuth nanorods etc.), and/or reporter enzymes or proteins. In a particular embodiment, the fluorescent imaging agent is DiD (DiIC18 (5); 1,1′-dioctadecyl-3,3,3′,3′-tetramethylindodicarbocyanine, 4-chlorobenzenesulfonate salt). While the molecular imaging agents may be biotinylated or conjugated/fused to avidin, streptavidin, or an analogue thereof for incorporation into VLPs, the molecular imaging agents may also be incorporated into the membrane of the VLPs without such modification. For example, molecular imaging agents which can be incorporated into membrane bilayers (e.g., fluorescent probes) can be delivered to VLP producing cells such that they are incorporated into the cellular membrane. Budding VLPs will then contain the incorporated molecular imaging agent in their membranes.

While the VLPs of the instant invention are generally described in terms of VLPs of HIV, the instant invention also encompasses VLPs of other viruses. In certain embodiments, the VLPs are of an enveloped virus. In certain embodiments, the VLPs are of a retrovirus or lentivirus. In certain embodiments, the VLPs comprise the structural proteins of the virus, but lack the viral genome as well as reverse transcriptase and integrase (if applicable). In certain embodiments, at least one of the structural proteins of the VLP (e.g., capsid and/or matrix protein) is biotinylated or conjugated/fused to avidin, streptavidin, or an analogue thereof, as described herein.

The instant invention also encompasses compositions (e.g., pharmaceutical compositions) comprising at least one VLP of the instant invention and at least one pharmaceutically acceptable carrier. As stated hereinabove, the VLP may comprise more than one therapeutic and/or molecular imaging agent. In a particular embodiment, the pharmaceutical composition comprises a first VLP comprising a first therapeutic and a second VLP comprising a second therapeutic, wherein the first and second therapeutics are different. The compositions (e.g., pharmaceutical compositions) of the instant invention may further comprise (e.g., not contained within the VLP) other therapeutic agents (e.g., other anti-HIV compounds).

In accordance with another aspect of the instant invention, methods of producing the VLPs of the instant invention are also provided. In certain embodiments, the method comprises delivering one or more expression vectors encoding the proteins of the VLPs (e.g., structural proteins and Env) to VLP producing cells. For example, the viral proteins of the VLP may be encoded by one or more plasmids and/or viral vectors (e.g., lentiviral vectors, adenoviral vectors). Examples of plasmids for the viral proteins include, but are not limited to: psPAX2 and pcDNA. In a particular embodiment, psPAX2 encoding Gag/Pro/Pol and pcDNA 89.6 envelope are delivered (e.g., transfected) to VLP producing cells to produce HIV VLPs. The methods further comprise delivering the therapeutic and/or molecule imaging agents to the VLP producing cells (e.g., at the same time as the structural proteins and/or before or after the structural proteins). The therapeutic and/or molecule imaging agent may be delivered to the cells as an expression vector encoding the therapeutic and/or molecule imaging agent (e.g., when it's a polypeptide). The therapeutic and/or molecule imaging agent may also be delivered to the VLP expressing cell. For example, the CRISPR ribonucleoprotein may be delivered to the VLP producing cells (e.g., electroporation, liposomes, etc.). Similarly, biotinylated or avidin, streptavidin or analogue thereof conjugated therapeutic and/or molecule imaging agent may be delivered to the VLP producing cells (e.g., electroporation, liposomes, etc.). As explained hereinabove, the method may further comprise expressing BirA in the cells to biotinylate proteins (e.g., by introduction of an expression vector encoding BirA. The methods can further comprise centrifuging (to exclude cell fractions); filtering culture supernatants (to exclude large extracellular vesicles; e.g., 0.22 μm filter), and/or ultracentrifuged through high-density liquid cushion to select for appropriately sized VLPs.

In certain embodiments, the VLP producing cells are mammalian cells. HIV-1 VLPs can be synthesized in mammalian cells to maintain glycosylation motifs essential to entry within CD4+ leukocytes. Small-scale batches (e.g., nanogram quantities) of VLPs can be synthesized through transient co-transfection of cells (e.g., HEK293T/FT cells) with packaging and envelope plasmids. Large-scale batches (e.g., microgram quantities or more) can be synthesized in bioreactors containing stable knock-in of VLP-encoding genes to mammalian lines. Examples of mammalian cells lines include, without limitation: HEK293, CHO, Vero, SV-1, 2BS, Mrc-5, RK-13, SP/20, BHK, L293, HeLa, or NIH3T3.

In accordance with another aspect of the instant invention, the VLPs of the instant invention may be used to deliver at least one therapeutic and/or molecular imaging agent to a cell or a subject (including non-human animals). The present invention also encompasses methods for preventing, inhibiting, and/or treating and/or tracking or monitoring (e.g., in real time) a viral infection, particularly an HIV infection. The methods comprise administering a VLP of the instant invention (optionally in a composition) to a subject in need thereof. The methods may further comprise (in the context of tracking and/or monitoring the viral infection) detecting the molecular imaging agent (e.g., in said subject). Monitoring and tracking the viral infection can also be used for tracking the effectiveness of a therapy. As explained herein, the VLPs of the present invention contain both a therapeutic and a molecular imaging agent and allows for both treating and imaging the viral infection.

Viral infections to be treated and/or monitored by the instant invention include, but are not limited to infections by: HIV, flavivirus, togaviruses, non-HIV retroviruses, lentiviruses, coronaviruses, orthomyxoviruses, paramyxovirus, rhabdoviruses, filoviruses, arenaviruses, bunyaviruses, and delta viruses. In a particular embodiment, the viral infection is a retroviral infection or a lentiviral infection. In a particular embodiment, the viral infection is a HIV infection.

The VLPs of the instant invention (optionally in a composition) can be administered to an animal, in particular a mammal, more particularly a human, in order to treat/inhibit/prevent the viral infection (e.g., a retroviral infection such as an HIV infection). The pharmaceutical compositions of the instant invention may also comprise at least one other therapeutic agent such as an antiviral agent, particularly at least one other anti-HIV compound/agent. The additional anti-HIV compound may also be administered in a separate pharmaceutical composition from the VLPs or compositions of the instant invention. The pharmaceutical compositions may be administered at the same time or at different times (e.g., sequentially).

The dosage ranges for the administration of the VLPs and/or compositions of the invention are those large enough to produce the desired effect (e.g., curing, relieving, treating, and/or preventing the viral infection (e.g., HIV infection), the symptoms of it (e.g., AIDS, ARC), or the predisposition towards it). The dosage should not be so large as to cause significant adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The VLPs described herein will generally be administered to a patient as a pharmaceutical composition. The term “patient” as used herein refers to human or animal subjects. These VLPs may be employed therapeutically, under the guidance of a physician.

The pharmaceutical compositions comprising the VLPs of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the VLPs in the chosen medium may be varied and the medium may be chosen based on the desired route of administration of the pharmaceutical composition. Except insofar as any conventional media or agent is incompatible with the VLPs to be administered, its use in the pharmaceutical composition is contemplated.

The dose and dosage regimen of VLPs according to the invention that are suitable for administration to a particular patient may be determined by a physician considering the patient's age, sex, weight, general medical condition, and the specific condition for which the VLPs are being administered and the severity thereof. The physician may also take into account the route of administration, the pharmaceutical carrier, and the VLP's biological activity.

Selection of a suitable pharmaceutical composition will also depend upon the mode of administration chosen. For example, the VLPs of the invention may be administered by direct injection or intravenously. In this instance, a pharmaceutical composition comprises the VLP dispersed in a medium that is compatible with the site of injection.

VLPs of the instant invention may be administered by any method. For example, the VLPs of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the VLP is administered parenterally. In a particular embodiment, the VLP is administered orally, intramuscularly, subcutaneously, or to the bloodstream (e.g., intravenously). In a particular embodiment, the VLP is administered intramuscularly or subcutaneously. Pharmaceutical compositions for injection are known in the art. If injection is selected as a method for administering the VLP, steps must be taken to ensure that sufficient amounts of the molecules or cells reach their target cells to exert a biological effect. Dosage forms for parenteral administration include, without limitation, solutions, emulsions, suspensions, dispersions and powders/granules for reconstitution.

Pharmaceutical compositions containing a VLP of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, oral, direct injection, intracranial, and intravitreal.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of VLPs may be determined by evaluating the toxicity of the molecules or cells in animal models. Various concentrations of VLPs in pharmaceutical composition may be administered to mice, and the minimal and maximal dosages may be determined based on the beneficial results and side effects observed as a result of the treatment. Appropriate dosage unit may also be determined by assessing the efficacy of the VLP treatment in combination with other standard drugs. The dosage units of VLPs may be determined individually or in combination with each treatment according to the effect detected.

The pharmaceutical composition comprising the VLPs may be administered at appropriate intervals until the pathological symptoms are reduced or alleviated, after which the dosage may be reduced to a maintenance level. The appropriate interval in a particular case would normally depend on the condition of the patient.

### Definitions

The following definitions are provided to facilitate an understanding of the present invention.

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “prodrug” refers to a compound that is metabolized or otherwise converted to a biologically active or more active compound or drug, typically after administration. A prodrug, relative to the drug, is modified chemically in a manner that renders it, relative to the drug, less active, essentially inactive, or inactive. However, the chemical modification is such that the corresponding drug is generated by metabolic or other biological processes, typically after the prodrug is administered.

The term “treat” as used herein refers to any type of treatment that imparts a benefit to a patient afflicted with a disease, including improvement in the condition of the patient (e.g., in one or more symptoms), delay in the progression of the condition, etc. In a particular embodiment, the treatment of a retroviral infection results in at least an inhibition/reduction in the number of infected cells and/or detectable viral levels.

As used herein, the term “prevent” refers to the prophylactic treatment of a subject who is at risk of developing a condition (e.g., HIV infection) resulting in a decrease in the probability that the subject will develop the condition.

A “therapeutically effective amount” of a compound or a pharmaceutical composition refers to an amount effective to prevent, inhibit, treat, or lessen the symptoms of a particular disorder or disease. The treatment of a microbial infection (e.g., HIV infection) herein may refer to curing, relieving, and/or preventing the microbial infection, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent production of viral particles, maturation of viral particles, viral attachment, viral uptake into cells, viral assembly, viral release/budding, viral integration, etc.

As used herein, the term “highly active antiretroviral therapy” (HAART) refers to HIV therapy with various combinations of therapeutics such as nucleoside reverse transcriptase inhibitors, non-nucleoside reverse transcriptase inhibitors, HIV protease inhibitors, and fusion inhibitors.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). “Hydrophobic” compounds are, for the most part, insoluble in water. As used herein, the term “hydrophilic” means the ability to dissolve in water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

An “antibody” or “antibody molecule” is any immunoglobulin, including antibodies and fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

As used herein, the term “targeting ligand” refers to any compound which specifically binds to a specific type of tissue or cell type, particularly without substantially binding other types of tissues or cell types. Examples of targeting ligands include, without limitation: proteins, polypeptides, peptides, antibodies, antibody fragments, hormones, ligands, carbohydrates, steroids, nucleic acid molecules, and polynucleotides.

The term “aliphatic” refers to a non-aromatic hydrocarbon-based moiety. Aliphatic compounds can be acyclic (e.g., linear or branched) or cyclic moieties (e.g., cycloalkyl) and can be saturated or unsaturated (e.g., alkyl, alkenyl, and alkynyl). Aliphatic compounds may comprise a mostly carbon main chain (e.g., 1 to about 30 carbons) and comprise heteroatoms and/or substituents (see below). The term “alkyl,” as employed herein, includes saturated or unsaturated, straight or branched chain hydrocarbons containing 1 to about 30 carbons in the normal/main chain. The hydrocarbon chain of the alkyl groups may be interrupted with one or more heteroatom (e.g., oxygen, nitrogen, or sulfur). An alkyl (or aliphatic) may, optionally, be substituted (e.g. with fewer than about 8, fewer than about 6, or 1 to about 4 substituents). The term “lower alkyl” or “lower aliphatic” refers to an alkyl or aliphatic, respectively, which contains 1 to 3 carbons in the hydrocarbon chain. Alkyl or aliphatic substituents include, without limitation, alkyl (e.g., lower alkyl), alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol. Aliphatic and alkyl groups having at least about 5 carbons in the main chain are generally hydrophobic, absent extensive substitutions with hydrophilic substituents.

The term “aryl,” as employed herein, refers to monocyclic and bicyclic aromatic groups containing 6 to 10 carbons in the ring portion. Examples of aryl groups include, without limitation, phenyl or naphthyl, such as 1-naphthyl and 2-naphthyl, or indenyl. Aryl groups may optionally include one to three additional rings fused to a cycloalkyl ring or a heterocyclic ring. Aryl groups may be optionally substituted through available carbon atoms with, for example, 1, 2, or 3 groups selected from hydrogen, halo, alkyl, polyhaloalkyl, alkoxy, alkenyl, trifluoromethyl, trifluoromethoxy, alkynyl, aryl, heterocyclo, aralkyl, aryloxy, aryloxyalkyl, aralkoxy, arylthio, arylazo, heterocyclooxy, hydroxy, nitro, cyano, sulfonyl anion, amino, or substituted amino. The aryl group may be a heteroaryl. “Heteroaryl” refers to an optionally substituted, mono-, di-, tri-, or other multicyclic aromatic ring system that includes at least one, and preferably from 1 to about 4, sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred.

The following example provides illustrative methods of practicing the instant invention and is not intended to limit the scope of the invention in any way.

### Example

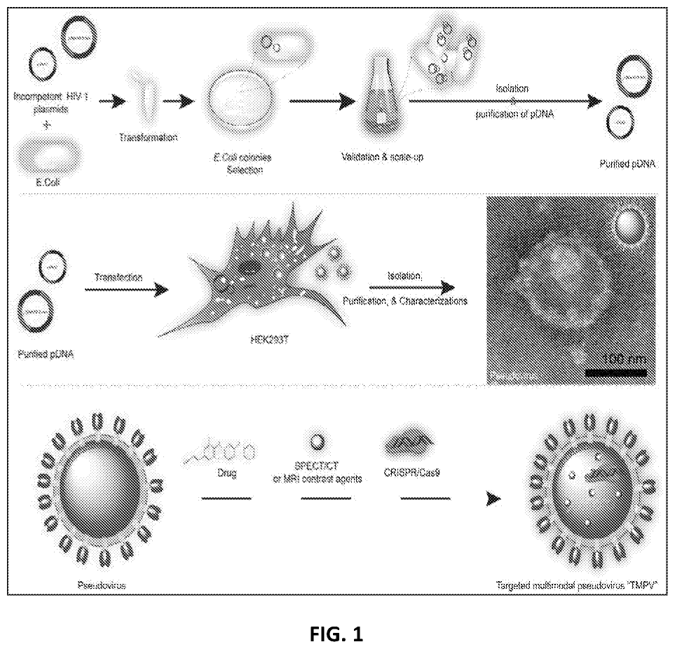

FIG. 1 provides a schematic for the synthesis of virus-like particles (VLPs). HIV-1 VLPs were manufactured by co-transfection of HEK293T with the packaging plasmid psPAX2 (NIH AIDS Reagent Program #11348) encoding HIV-1 Gag/Pro/Pol and pcDNA encoding HIV-1 envelope proteins. The plasmid pcDNA was derived from the 89.6 env gene that is both CCR5 (R5) and CXCR4 (X4) tropic. The created pseudotyped HIV-1_{89.6} VLPs were labeled with fluorescent DiD (DiD (DiIC18 (5); 1,1′-dioctadecyl-3,3,3′,3′-tetramethylindodicarbo-cyanine, 4-chlorobenzenesulfonate salt) dye or loaded with antiretroviral drug (ARV) (rilpivirine, RPV), radiolabeled or encased with heavy metals to create a multimodal nanoparticle (¹¹¹In/¹⁷⁷Lu/⁹⁹mTC/⁶⁴Cu/¹³¹I, iron oxide or cobalt ferrite, other metals), or with reporter genes and drugs. The activity of each or all of these payloads was detectable by flow cytometry and SPECT/CT radiography. The dual R5/X4-tropic VLPs were identified to improve targeting and delivery. These particles can reach monocyte-macrophages and CD4+ T cell populations for delivery of cargos to cells that harbor virus in latent or productive manners. Together, the data provided here show the impact of the VLP invention for delivery of therapeutic agents that combat HIV-1 infection or used to eliminate virus itself. As VLPs target the same cell populations as replication competent HIV-1, they can be deployed as a “best” therapeutic carrier. Testing in humanized mice show that 84% of CD14+ monocyte-macrophages and 36% of CD4+ T cells are VLP targets with limited off-target signals or toxicities noted.

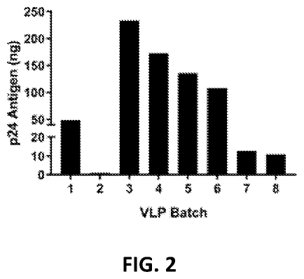

As explained above, the HIV-1 VLPs were synthesized by co-transfecting plasmids encoding HIV-189.6 envelope with lentiviral packaging proteins in HEK293T cells. Concentrated VLPs were characterized by transmission electron microscopy (TEM) for morphology and physical properties were assessed by dynamic light scattering (DLS). The VLPs were generally spherical and had a diameter of about 100 to 150 nm by TEM (see FIG. 1). DLS indicated that the average size of the VLPs was 102.5±8.3, the polydispersity index was 0.412±0.033, and the zeta potential was −15.6±0.9 mV. The presence of gp120 and p24 in the VLPs was confirmed by Western blot. p24 antigenicity was also determined by ELISA (FIG. 2), demonstrating the yield of VLPs.

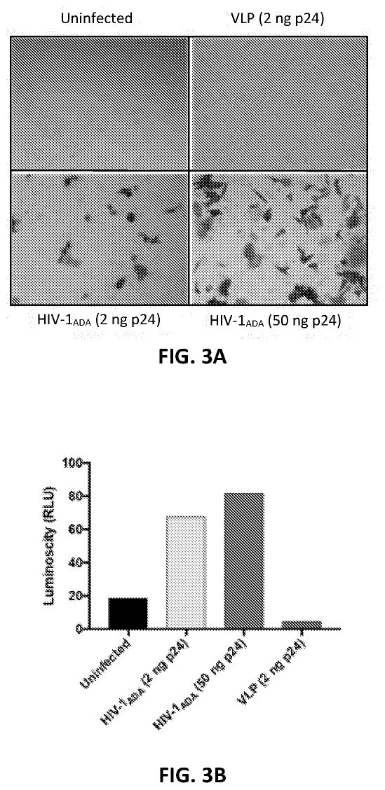

The replication incompetency of the HIV-1 VLPs was then tested with TZM-bl reporter cells. The TZM-bl cell line enables quantitative analysis of HIV using β-gal and/or luciferase as a reporter. The cells express large amounts of CD4 and CCR5 and constitutively express CXCR4. The cells also possess copies of the luciferase and β-galactosidase genes under control of the HIV-1 Tat promoter. TZM-bl reporter cells were challenged with HIV-1ADA or VLP. Two days post-challenge, cells were incubated with X-gal substrate which produces blue pigment in the presence of β-galactosidase (FIG. 3A) or D-luciferin substrate that yields luminescence in the presence luciferase expressed due to the presence of HIV Tat protein (FIG. 3B). As seen in FIG. 3, the HIV-1 VLPs are replication incompetent.

VLP loading and membrane labeling with tracking agents was then performed. Red quantum dots (rQD) were synthesized from cadmium-selenium cores and layered with cadmium-sulfide mantles. rQD typically have a diameter less than 10 nm and demonstrate red emission under ultraviolet (UV) excitation. A VLP lipofection-based technique was used to load rQD. The loading of rQD into VLP was confirmed by UV imaging of agarose gel electrophoresis of VLP versus VLP loaded with rQD. In order to load DiD (1,1′-dioctadecyl-3,3,3′,3′-tetramethylindodicarbocyanine, 4-chlorobenzenesulfonate salt), HEK293T cells were pretreated with DiD (300 μg) prior to transfection with the VLP producing plasmids. The resultant VLP (VLP-DiD) possessed envelope derived from producer cell plasma membrane, thereby containing DiD. DiD was readily visible by microscopy of producer HEK293T cells. DID was also readily visible in crude culture supernatant but not 0.22 μm filtered supernatant. The VLP-DiD was separated from free DiD by ultracentrifugation (135,000×g, 4 hours) of filtered supernatant through a 20% sucrose gradient.

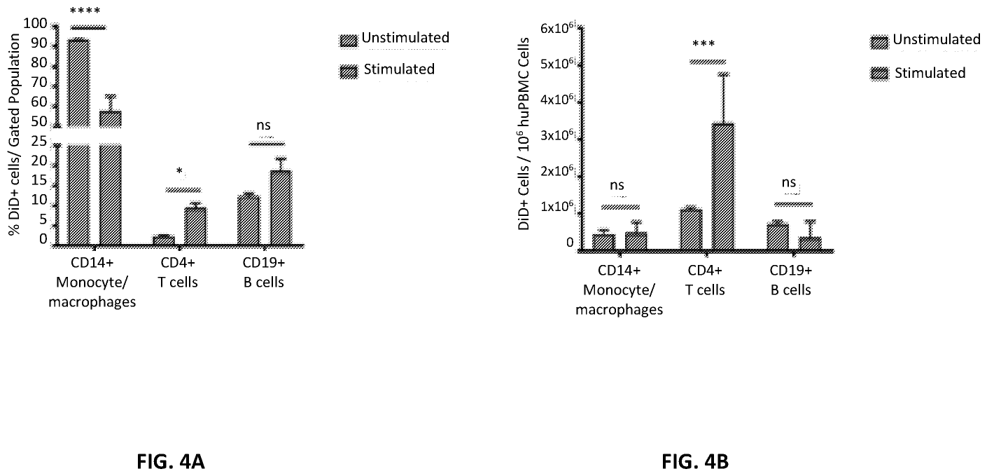

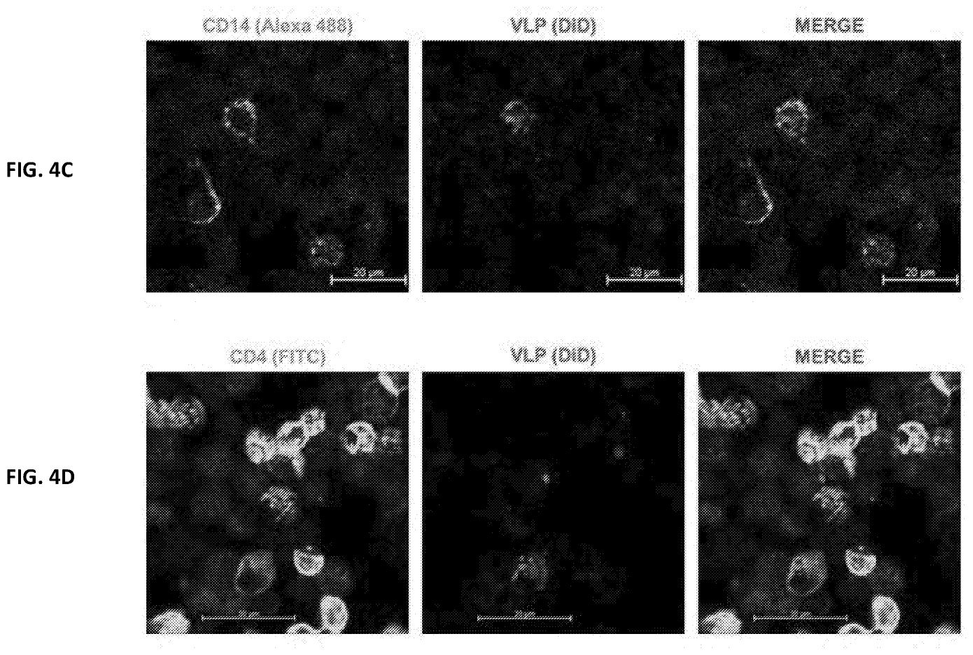

The ability of the HIV-1 VLPs to target HIV-infectible cells in vitro was then tested. Human peripheral blood mononuclear cells (PBMCs) were cultured in the absence or presence of IL2 (20 U/mL) plus phytohemagglutinin (PHA, 40 μM/mL) immune stimulant for 3 days. PHA and IL-2 exposure primes PBMCs for HIV infection. The treated PBMCs were then treated with DiD fluorescently-labeled VLPs (2 ng p24/1×10⁶ cells) in biological triplicates for 24 hours. Thereafter, treated cells were antibody stained for on-target CD14⁺ monocyte/macrophages and CD4⁺ T cells or off-target CD19⁺ B cells and subjected to flow cytometry. The percent of gated populations positive for DiD fluorescent label (FIG. 4A) and subpopulations normalized by relative abundance (FIG. 4B) were plotted (mean±SD). Confocal microscopy images were also taken of unstimulated PBMCs treated with VLP-DiD (750 ng p24/2×10⁶ cells, 1 hour) followed by immunostaining anti-CD14-Alexa488 (FIG. 4C) or anti-CD4-FITC (FIG. 4D) antibodies for 30 minutes. As seen in FIG. 4, CD4⁺ T cells readily were infected by the HIV-1 VLPs.

The ability of the HIV-1 VLPs to target HIV-infectible cells in vivo was then tested. Human CD34⁺ hematopoietic stem-cell reconstituted NSG (humanized) mice were made as follows. NSG (NOD.Cg-Prkdc^{scid} Il2rgt^{m1Wjl}/SzJ) mice were obtained from the Jackson Laboratories, Bar Harbor, Me. CD34+ HSC were enriched from human cord blood or fetal liver cells using immune-magnetic beads (CD34+ selection kit; Miltenyi Biotec Inc., Auburn, Calif.). CD34+ cell purity was >90% by flow cytometry. Cells were transplanted into newborn mice irradiated at 1 Gy using a RS-2000×-Ray Irradiator (Rad Source Technologies, Buford, Ga.). Cells were transplanted by intrahepatic (i.h.) injection of 50,000 cells/mouse in 20 μl phosphate-buffered saline (PBS) with a 30-gauge needle. Humanization of the animals was affirmed by flow cytometry for the presence of human CD45 and CD3 positive blood immune cells (Gorantla, et al. (2010) Am. J. Pathol., 177:2938-2949; O'Doherty, et al. (2000) J. Virol., 74:10074-10080).

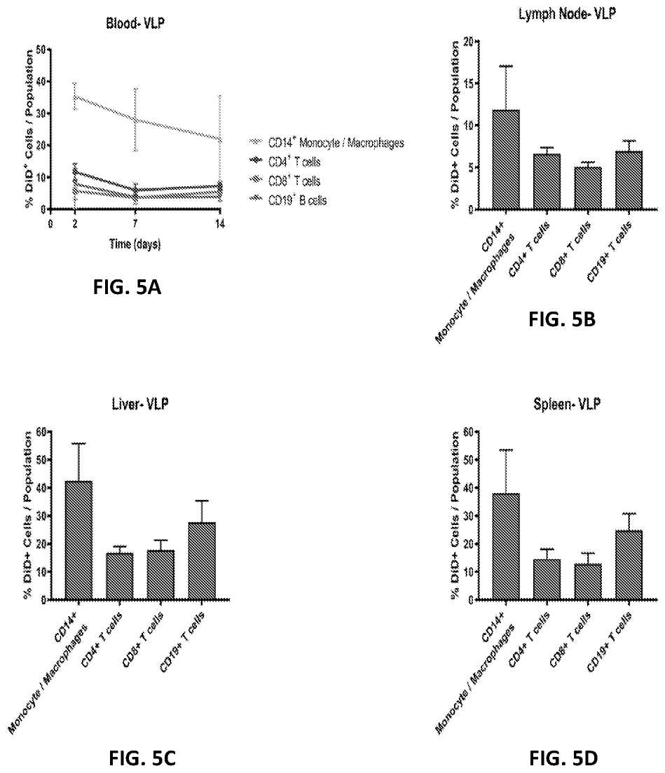

Humanized mice were treated with VLP-DiD (50 ng p24/mouse, i.v.) in triplicate. The mice were bled (days 2, 7) and sacrificed 14 days post-treatment. Blood as well as single-cell suspensions from lymph nodes, liver, and spleen were subjected to flow cytometry. As seen in FIG. 5, HIV-1 VLPs targeted HIV-infectible cells in vivo.

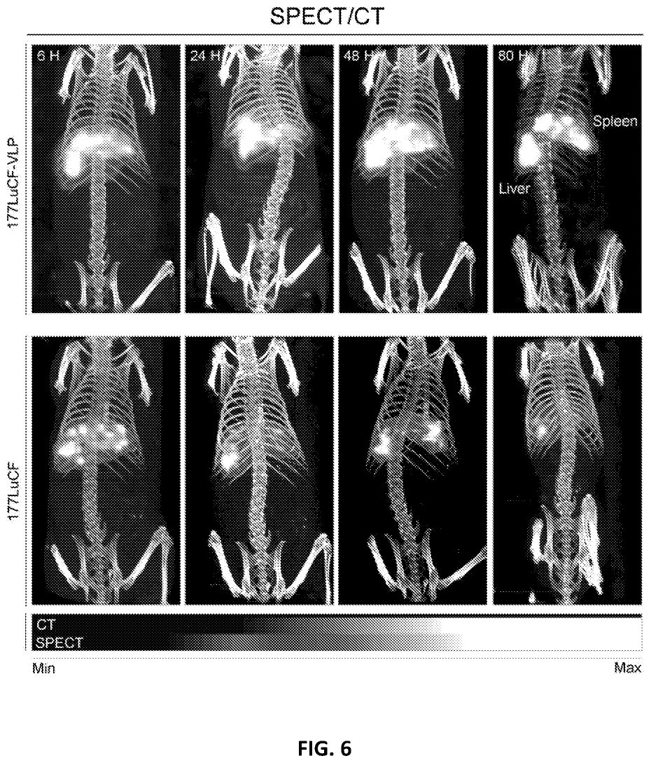

Real time biodistribution tests were also performed in the humized mice. Single photon emission computed tomography computerized tomography (SPECT/CT) imaging of intrinsic labeling of ¹⁷⁷Lu into CFEu. ¹⁷⁷Lu-CF-VLP and ¹⁷⁷Lu-CF nanoparticles (˜7000 μCi, particle size of ˜150 nm) were intravenously injected into a humanized mouse. Whole body SPECT/CT images were collected at 6, 12, 24, 48, 80, and 120 hours after injection (FIG. 6). ¹⁷⁷Lu labeled intensity is reflective of the key provided. Anatomically (by CT scan), high signal intensity was detected in the liver and spleen. The images were acquired over sixty-four projections at 20 seconds/projection. The detector radius of rotation was set at 47 mm to provide a pixel size of −60 mm. A multi-pinhole N5F75A10 collimator, mouse style, 1 mm aperture was used to acquire the CT images (Flex Triumph platform, TriFoil Imaging, Chatsworth, Calif.). The images were adjusted for an appropriate fitting with the tracer distribution.

Modified VLPs were also synthesized. Briefly, the genes encoding reverse transcriptase and integrase were removed by restriction enzyme digestion. Further, the sequence encoding for the AviTag was added to the gene encoding p17 and the sequence encoding for monomeric streptavidin (mSA) was added to the gene encoding p24. These modification were made to plasmid constructs with significant portions of Pol deleted, with the resultant VLPs lacking reverse transcriptase activity.

Maxividin binds biotin and biotin conjugates with high affinity. Maxividin is an optimized monomeric streptavidin. The interaction between biotin and biotinylated cabotegravir (BCAB) ligands and the maxividin active site were predicted in silico using Swiss-Doc server and visualized in Biovia Discovery Studio. The affinity of maxividin-ligand interactions was calculated using Swiss-Doc server and compared on the basis of binding free energy. Streptavidin binds biotin with a binding free energy of −10 kcal/mol and monomeric streptavidin binds biotin with a binding free energy of −7.2 kcal/mol. In contrast, maxividin binds biotin with a binding free energy of −7.9 kcal/mol and binds BCAB with a binding free energy of −10.37 kcal/mol.

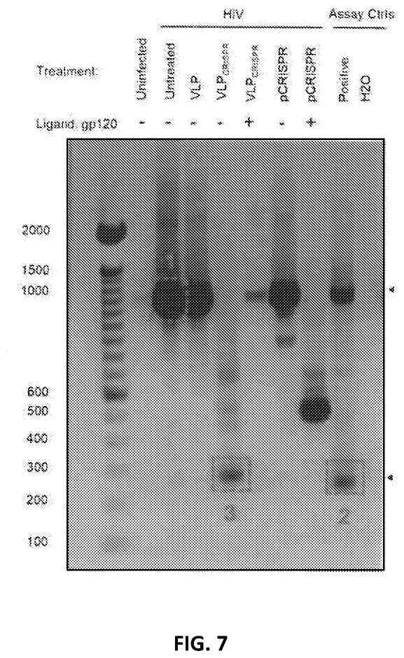

The ability of the VLPs to excise HIV-1 proviral DNA was also tested. CEM-SS T cells were infected with HIV-1_{NL4-3} (multiplicity of infection (MOI) 0.05) for 24 hours and then washed 3× with PBS to remove virus. After 7 days of infection, cells were treated with CRISPR-encoding plasmid (pCRISPR; 1 μg/10⁶ cells via lipofection), VLPs (unloaded control), or CRISPR-delivering VLPs (VLP_{CRISPR}) at 5 ng p24 per 10⁶ cells. As a control for specificity to CD4+ cells, excess recombinant HIV-1 gp120 (1 μg/10⁶ cells) was added during treatment. DNA was extracted 72 hours after treatment and analyzed by PCR for excision of nucleotides between the 5′LTR and gag sequences. As seen in FIG. 7, VLPCRISPR excised the HIV-1 proviral DNA and this effect could be blocked by the addition of gp120.

The ability of the HIV-1 VLPs to target HIV-infectable cells in vitro was also tested. Human PBMCs were cultured in the absence or presence of IL2 and PHA immune stimulants for three days. PBMCs were then treated with DiD fluorescently-labeled VLPs in biological triplicates and subjected to flow cytometry. The percent of gated populations positive for DiD fluorescent label and subpopulations normalized by relative abundance was plotted. Statititcal analyses were performed using 2-way ANOVA. As seen in FIG. 4A-4B, HIV-1 VLPs targeted HIV-infectable cells in vitro. Representative confocal microscopy images of monocyte-macrophages and CD4+ cells treated with VLP-DiD are provided in FIGS. 4C and 4D, respectively. HIV-1 VLPs target HIV-infectible leukocytes through gp120-to-CD4 mediated binding. Interactions between VLPs and target cells can be abrogated through addition of competitive ligands (e.g. recombinant gp120, MIP1, CCL3, CCL4, CCL3L1, CXCL12).

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A human immunodeficiency virus (HIV) virus-like particle (VLP), wherein said VLP comprises at least one HIV structural protein and HIV envelope protein, wherein said VLP does not contain the HIV genome and lacks reverse transcriptase and integrase.

2. The VLP of claim 1, wherein said VLP comprises HIV Gag, Pro, gp120, and gp41.

3. The VLP of claim 2, wherein said Gag is cleaved into at least matrix and capsid.

4. The VLP of claim 1, wherein said HIV envelope protein is dual-tropic for CCR5 and CXCR4.

5. The VLP of claim 4, wherein said HIV envelope protein is from HIV-1_{89.6}.

6. The VLP of claim 1, wherein said VLP comprises at least one therapeutic and/or at least one molecular imaging agent.

7. The VLP of claim 6, wherein the VLP comprises at least one a therapeutic and at least one molecular imaging agent.

8. The VLP of claim 6, wherein at least one therapeutic and/or at least one molecular imaging agent is biotinylated.

9. The VLP of claim 6, wherein at least one therapeutic and/or at least one molecular imaging agent is conjugated to monomeric streptavidin or an analogue thereof.

10. The VLP of claim 6, wherein said therapeutic is an anti-HIV agent.

11. The VLP of claim 6, wherein said therapeutic is a CRISPR ribonucleoprotein, wherein the guide RNA of the CRISPR ribonucleoprotein targets the HIV genome.

12. The VLP of claim 6, wherein said VLP comprises an anti-HIV agent and a CRISPR ribonucleoprotein.

13. The VLP of claim 1, wherein said VLP comprises at least one therapeutic.

14. The VLP of claim 1, wherein said VLP comprises a biotinylated HIV structural protein.

15. The VLP of claim 14, wherein said biotinylated HIV structural protein is biotinylated matrix.

16. The VLP of claim 1, wherein said VLP comprises a HIV structural protein conjugated to avidin, streptavidin, or an analogue thereof.

17. The VLP of claim 16, wherein said VLP comprises capsid conjugated to monomeric streptavidin or an analogue thereof.

18. The VLP of claim 16, wherein said VLP further comprises a biotinylated therapeutic.

19. The VLP of claim 14, wherein said VLP further comprises a therapeutic conjugated to monomeric streptavidin or an analogue thereof.

20. The VLP of claim 14, wherein said VLP further comprises a HIV structural protein conjugated to avidin, streptavidin, or an analogue thereof.

21. The VLP of claim 20, wherein said VLP further comprises a biotinylated therapeutic and/or a therapeutic conjugated monomeric streptavidin or an analogue thereof.

22. A method of synthesizing a VLP of any one of claims 1 to 21, said method comprising expressing said HIV structural proteins and envelope protein in mammalian cells.

23. A method of monitoring a viral infection, said method comprising administering at least one VLP of any one of claims 1 to 21 to a subject and detecting the presence of the molecular imaging agent.

24. A method of treating, inhibiting, and/or preventing a viral infection, said method comprising administering at least one VLP of any one of claims 1 to 21 to a subject in need thereof.

25. The method of claim 24, wherein said viral infection is an HIV infection.


SEQUENCE LISTING (4 sequences)

SEQ ID NO: 1 - protein, 15 aa, Artificial Sequence
  biotinylation tag
GLNDIFEAQKIEWHE

SEQ ID NO: 2 - protein, 125 aa, Artificial Sequence
  monomeric streptavidin
EFASAEAGITGTWYNQHGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGS

SEQ ID NO: 3 - protein, 114 aa, Artificial Sequence
  monomeric streptavidin
AEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVK

SEQ ID NO: 4 - protein, 125 aa, Artificial Sequence
  maxavidin
EFASAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKPSAASGS